(19) United States
(12) Patent Application Publication
(10) Pub. No.: US 2010/0221323 A1
(43) Pub. Date: Sep. 2, 2010

(54) Crystal Entecavir Formulation And The Preparation Method Thereof

(75) Inventors: Deping Yi, Huancheng Donglu (CN); Zhike Tian, Huancheng Donglu (CN); Weidong Ye, Huancheng Donglu (CN)

Correspondence Address: WILLIAM D. HARE, 66 WITHERSPOON STREET, STE. 1, PMB 317, PRINCETON, NJ, 08542-9944, US

(73) Assignee: Zhejiang Medicine Co., Ltd. Xinchang Pharmaceutical Factory, Huancheng Donglu, Zhejiang Province (CN)

(21) Appl. No.: 12/674,879

(22) PCT Filed: Aug. 25, 2008
(86) PCT No.: PCT/CN08/01523

§ 371 Date: Feb. 23, 2010

(30) Foreign Application Priority Data
Aug. 23, 2007 (CN) CN200710143096.7

(51) Int. Cl. A61K 9/48; A61K 31/522; A61K 9/28; A61P 31/20

(52) U.S. Cl. 424/452; 514/263.37; 424/465

(57) ABSTRACT

The present invention relates to a pharmaceutical composition for treating hepatitis B virus infection, comprising the crystal entecavir as pharmaceutically active ingredients and pharmaceutically acceptable excipients. The tablet and capsule of the pharmaceutical composition have more stronger stabilization than that of amorphous entecavir under conditions of lighting, high temperature and high humidity.

### FIELD OF THE INVENTION

The present invention relates to a crystal entecavir formulation, and its clinical use in treatment of hepatitis B virus infection.

### BACKGROUND OF THE INVENTION

Chronic hepatitis B virus infection is one of the most severe liver diseases in morbidity and death rate in the worldwide range. At present, pharmaceuticals for treating chronic hepatitis B (CHB) virus infection are classified to interferon α and nucleoside/nucleotide analogue, i.e. Lamivudine and Adefovir. However, these pharmaceuticals can not meet needs for doctors and patients in treating chronic hepatitis B virus infection because of their respective limitation. Entecavir (ETV) is referred to as 2′-cyclopentyl deoxyguanosine (BMS2000475) which belongs to analogues of Guanine nucleotide and is phosphorylated to form an active triple phosphate in vivo. The triple phosphate of entecavir inhibits HBV polymerase by competition with 2′-deoxyguanosine-5′-triphosphate as a nature substrate of HBV polymerase, so as to achieve the purpose of effectively treating chronic hepatitis B virus infection and have strong anti-HBV effects. Entecavir, [1S-(1α,3α,4β]-2-amino-1,9-dihydro-9-[4-hydroxy-3-hydroxymethyl]-2-methylenecyclopentyl]-6H-purin-6-one, monohydrate, and has the molecular formula of C₁₂H₁₅N₅O₃.H₂O and the molecular weight of 295.3. Its structural formula is as follows:

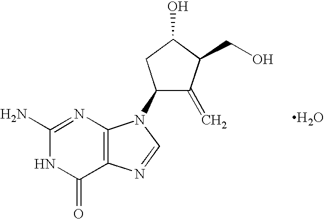

Entecavir is successfully developed by Bris-Tol-Myers Squibb Co. of USA firstly and the trademark of the product formulation is Baraclude™, including two types of formulations of tablet and oral solution having 0.5 mg and 1 mg of dosage. The Chinese publication No. CN1310999 made by COLONNO, Richard, J. et al discloses a low amount of entecavir and uses of the composition containing entecavir in combination with other pharmaceutically active substances for treating hepatitis B virus infection, however, the entecavir is non-crystal. In addition, its oral formulations such as tablet and capsule are made by a boiling granulating process. The process is too complicated to control quality of products during humidity heat treatment even though ensuring uniform distribution of the active ingredients.

The present inventors of the Chinese patent application CN200710004988.9 provides a crystal entecavir in an aqueous solution as active ingredients. It has been found that tablets and capsules produced by a crystal entecavir in an aqueous solution as active ingredients have more stronger stabilization than that of amorphous entecavir under conditions of lighting, high temperature and high humidity.

The object of the present invention is to provide an oral formulation of the composition containing a crystal entecavir and the method of preparing for the same. The method comprises uniformly mixing the active crystal entecavir with glidants (or lubricants), and then uniformly mixing with diluents, adhesives, disintegrants and lubricants, and then directly tabletting or filling capsule. Comparing with processes of wet granulation, boiling granulating or spray drying granulation, the method is more simply in operation, more suitable for industrialized production and control of product quantity, reduced energy consumption, and reduced production cost.

### SUMMARY OF THE INVENTION

The present invention provides a pharmaceutical composition comprising a crystal entecavir and its use for treatment of hepatitis B virus infection. The composition comprises an amount of from 0.001 mg to 25 mg of a crystal entecavir, preferably an amount of from 0.01 mg to 10 mg of a crystal entecavir, more preferably a amount of from 0.1 mg to 5 mg of a crystal entecavir. The composition is administered for once daily to treat hepatitis B virus infection in an adult human patient upon clinical applications.

The present invention further provides a pharmaceutical composition for oral administrations comprising a crystal entecavir as active ingredients. The active ingredient of the entecavir formulation product for sold is now an amorphous entecavir. However, the crystal entecavir in aqueous solutions used as active ingredients of the present invention has been filed by the present inventors (the Chinese patent application No. 200710004988.9).

The present invention further provides a pharmaceutical composition comprising the crystal entecavir, which can be formulated for administration by any suitable means. For example, compositions for oral administration, which are preferred, but not limited, can be in the form of tablets, capsules, granules or powders, in which the tablets and capsules is most preferred.

The present invention further provides a method of preparing for a pharmaceutical composition comprising the crystal entecavir in the form of tablets and capsules, but not directly limited process of tabletting or filling, wet granulation, boiling granulating or spray drying granulation, and process of directly tabletting or filling is preferred. The process of directly tabletting or filling comprises uniformly mixing the active crystal entecavir with glidants (or lubricants), then uniformly mixing with diluents, adhesives, and disintegrants, and then directly tabletting or filling capsule. Comparing with process of wet granulation, boiling granulating or spray drying granulation, the present method is more simply in operation, more suitable for industrialized production and control of product quantity, reduced energy consumption, and reduced production costs.

The technical solution is as follows:

The present invention provides a pharmaceutical composition for treating hepatitis B virus infection, comprising the crystal entecavir as active ingredients and pharmaceutically acceptable excipients.

The pharmaceutical composition comprises an amount of from 0.001 mg to 25 mg of the crystal entecavir, preferably from 0.01 mg to 10 mg, more preferably from 0.01 mg to 5 mg.

According to the present invention, the pharmaceutically acceptable excipients comprise diluents in an amount of from 50% to 90% by weight of the total composition, and the diluents adheres with the crystal entecavir by an adhesive.

The diluents include one or more compounds selected from lactose, starch, microcrystalline cellulose, sucrose, glucose, mannitol, xylitol, maltitol, dextrin, calcium sulfate and calcium phosphate, in which lactose, starch and microcrystalline cellulose are preferred.

According to the present invention, the adhesives include one or more compounds selected from povidone, hydroxypropylmethylcellulose, alginic acid, sodium alginate, carbomer, poloxamer, and gelatin, in which povidone is preferred. The adhesive is present in an amount of from 2% to 18% by weight of the total composition.

According to the present invention, the pharmaceutical composition further comprises glidants and disintegrants.

Said glidants include one or more compounds selected from silica, stearic acid, magnesium stearate, sodium stearate, calcium stearate, sodium lauryl sulfate, sucrose fatty acid ester, and talcum powder, in which silica and magnesium stearate are preferred. Said glidant is present in an amount of from 0.1% to 5% by weight of the total composition.

Said disintegrants include one or more compounds selected from sodium carboxymethyl starch, hydroxypropyl cellulose, croscarmellose sodium, crospovidone, and sodium starch glycolate, in which sodium carboxymethyl starch is preferred. Said disintegrant is present in an amount of from 1% to 5% by weight of the total composition.

In addition, the pharmaceutical composition is formulated in the form for administration by any suitable means. Preferably, the pharmaceutical composition is formulated in the form of tablets and capsules.

Tablets and capsules of crystal entecavir can respectively be produced by directly tabletting or filling, and then the tablets are further coated so as to have outer film coating. The coating may be sugar coating or film coating, and the film coating is preferred. Suitable materials for use in the film coating are coating agents, light-screening agents, pigments, plasticizers, solubilizing agents, etc. The coating agents include hydroxypropylmethyl cellulose, hydroxyethyl cellulose, hydroxypropyl cellulose, hydroxypropyl methylcellulose phthalate, hypromellose phthalate etc., and hydroxypropyl methyl cellulose is preferred. The light-screening agents include titanium dioxide. The pigments include various of iron oxides. The plasticizers include polyethylene glycol. Solubilizing agents include polysorbate 80. The above coating ingredients are dispersed in a suitable solvent, preferably water. The coating ingredients can be applied to the tables using conventional pan coating techniques.

In addition, the present invention also provides a method of preparing for the pharmaceutical composition, which comprises the following steps:

(1) sieving the crystal entecavir as a pharmaceutically active ingredient and the glidant through 120 mesh screen and mixing them together to obtain a mixture;

(2) uniformly mixing the mixture of Step (1) with the diluent, adhesive and disintegrant, and then compressing it into tablets or filling into capsules.

In addition, the present invention further provides uses of the pharmaceutical composition in preparing for pharmaceuticals of treating hepatitis B virus infection.

It has been found from experiments that the tablet and capsule of the pharmaceutical composition produced by the crystal entecavir in an aqueous solution as active ingredients have more stronger stabilization than that of amorphous entecavir under conditions of lighting, high temperature and high humidity.

### DETAILED DESCRIPTION AND PREFERRED EMBODIMENTS THEREOF

Hereafter, the crystal entecavir formulations of the present invention will be described specifically with reference to examples. The examples are given only for illustration of the technical solution of the present invention and should not be construed to limit the present invention.

### Example 1

| Ingredients: | | |
|---|---|---|
| Crystal entecavir | 0.001 | g |
| Stearic acid | 1.999 | g |
| Sucrose | 50 | g |
| Calcium sulfate | 40 | g |
| Poloxamer | 3 | g |
| Hydroxypropyl cellulose | 5 | g |

1000 tablets (weight: 100 mg per tablet) are produced.

Preparation method is as follows:

The crystal entecavir and stearic acid are respectively sieved through 120 mesh screen and uniformly mixed to obtain a mixture, then the mixture is uniformly mixed with sucrose, calcium sulfate, poloxamer and hydroxypropyl cellulose, and then compressed into a tablet.

### Example 2

| Ingredients: | | |
|---|---|---|
| Crystal entecavir | 0.01 | g |
| Silica | 0.05 | g |
| Magnesium stearate | 0.05 | g |
| Lactose | 89.89 | g |
| Povidone | 5 | g |
| Sodium carboxymethyl starch | 5 | g |

1000 tablets (weight: 100 mg per tablet) are produced.

Preparation method is as follows:

The crystal entecavir, silica and magnesium stearate are respectively sieved through 120 mesh screen and uniformly mixed to obtain a mixture, then the mixture is uniformly mixed with lactose, povidone and sodium carboxymethyl starch, and then compressed into a tablet.

### Example 3

| Ingredients: | | |
|---|---|---|
| Crystal entecavir | 0.1 | g |
| Sucrose fatty acid ester | 5 | g |
| Starch | 81.9 | g |
| Alginic acid | 8 | g |
| Hydroxypropyl cellulose | 2 | g |
| Sodium starch glycolate | 3 | g |

1000 tablets (weight: 100 mg per tablet) are produced.

Preparation method is as follows:

The crystal entecavir and sucrose fatty acid ester are respectively sieved through 120 mesh screen and uniformly mixed to obtain a mixture, then the mixture is uniformly mixed with starch, alginic acid, hydroxypropyl cellucose and sodium starch glycolate, and then compressed into a tablet.

### Example 4

| Ingredients: | | |
|---|---|---|
| Crystal entecavir | 0.5 | g |
| Sodium stearate | 3 | g |
| Lactose | 45 | g |
| Starch | 38.5 | g |
| Hydroxypropylmethyl cellulose | 10 | g |
| Crospovidone | 3 | g |

1000 tablets (weight: 100 mg per tablet) are produced.

Preparation method is as follows:

The crystal entecavir and sodium stearate are respectively sieved through 120 mesh screen and uniformly mixed to obtain a mixture, then the mixture is uniformly mixed with lactose, starch, hydroxypropylmethyl cellulose and crospovidone, and then compressed into a tablet.

### Example 5

| Ingredients: | | |
|---|---|---|
| Crystal entecavir | 0.5 | g |
| Silica | 1.5 | g |
| Magnesium stearate | 1.5 | g |
| Lactose | 25 | g |
| Starch | 30 | g |
| Microcrystalline cellulose | 34.5 | g |
| Povidone | 6 | g |
| Sodium carboxymethyl starch | 3 | g |

1000 tablets (weight: 100 mg per tablet) are produced.

Preparation method is as follows:

The crystal entecavir, magnesium stearate and silica are respectively sieved through 120 mesh screen and uniformly mixed to obtain a mixture, then the mixture is uniformly mixed with lactose, starch, microcrystalline cellulose, povidone and sodium carboxymethyl starch, and then compressed into a tablet.

### Example 6

| Ingredients: | | |
|---|---|---|
| Crystal entecavir | 1 | g |
| Silica | 1.5 | g |
| Magnesium stearate | 1.5 | g |
| Starch | 84 | g |
| Povidone | 10 | g |
| Sodium carboxymethyl starch | 2 | g |

1000 tablets (weight: 100 mg per tablet) are produced.

Preparation method is as follows:

The crystal entecavir, silica and magnesium stearate are respectively sieved through 120 mesh screen and uniformly mixed to obtain a mixture, then the mixture is uniformly mixed with starch, povidone and sodium carboxymethyl starch, and then compressed into a tablet.

### Example 7

| Ingredients: | | |
|---|---|---|
| Crystal entecavir | 1.5 | g |
| Sodium lauryl sulfate | 5 | g |
| Microcrystalline cellulose | 87.5 | g |
| Carbomer | 4 | g |
| Croscarmellose sodium | 2 | g |

1000 tablets (weight: 100 mg per tablet) are produced.

Preparation method is as follows:

The crystal entecavir and sodium lauryl sulfate are respectively sieved through 120 mesh screen and uniformly mixed to obtain a mixture, then the mixture is uniformly mixed with microcrystalline cellulose, carbomer and croscarmellose sodium, and then compressed into a tablet.

### Example 8

| Ingredients: | | |
|---|---|---|
| Crystal entecavir | 2 | g |
| Magnesium stearate | 3 | g |
| Lactose | 38 | g |
| Microcrystalline cellulose | 50 | g |
| Povidone | 2 | g |
| Sodium carboxymethyl starch | 5 | g |

1000 tablets (weight: 100 mg per tablet) are produced.

Preparation method is as follows:

The crystal entecavir and magnesium stearate are respectively sieved through 120 mesh screen and uniformly mixed to obtain a mixture, then the mixture is uniformly mixed with lactose, microcrystalline cellulose, povidone and sodium carboxymethyl starch, and then compressed into a tablet.

### Example 9

| Ingredients: | | |
|---|---|---|
| Crystal entecavir | 5 | g |
| Silica | 5 | g |
| Microcrystalline cellulose | 71 | g |
| Povidone | 18 | g |
| Sodium carboxymethyl starch | 1 | g |

1000 tablets (weight: 100 mg per tablet) are produced.

Preparation method is as follows:

The crystal entecavir and silica are respectively sieved through 120 mesh screen and uniformly mixed to obtain a mixture, then the mixture is uniformly mixed with microcrystalline cellulose, povidone and sodium carboxymethyl starch, and then compressed into a tablet.

### Example 10

| Ingredients: | | |
|---|---|---|
| Crystal entecavir | 10 | g |
| Sodium stearate | 2 | g |
| Talcum powder | 2 | g |
| Glucose | 30 | g |
| Calcium phosphate | 40 | g |
| Hydroxypropylmethyl cellulose | 11 | g |
| Sodium starch glycolate | 5 | g |

1000 tablets (weight: 100 mg per tablet) are produced.

Preparation method is as follows:

The crystal entecavir, sodium stearate and talcum powder are respectively sieved through 120 mesh screen and uniformly mixed to obtain a mixture, then the mixture is uniformly mixed with glucose, calcium phosphate, hydroxypropylmethyl cellulose and sodium starch glycolate, and then filled into a No. 4 size of capsule.

### Example 11

| Ingredients: | | |
|---|---|---|
| Crystal entecavir | 15 | g |
| Stearic acid | 4 | g |
| Xylitol | 43 | g |
| Maltitol | 30 | g |
| Gelatin | 6 | g |
| Croscarmellose sodium | 2 | g |

1000 tablets (weight: 100 mg per tablet) are produced.

Preparation method is as follows:

The crystal entecavir and stearic acid are respectively sieved through 120 mesh screen and uniformly mixed to obtain a mixture, then the mixture is uniformly mixed with xylitol, maltitol, gelatin and croscarmellose sodium, and then filled into a No. 4 size of capsule.

### Example 12

| Ingredients: | |
|---|---|
| Crystal entecavir | 25 g |
| Sodium lauryl sulfate | 2.5 g |
| Sucrose fatty acid ester | 2.5 g |
| Mannitol | 30 g |
| Dextrin | 20 g |
| Alginic acid | 6 g |
| Sodium alginate | 9 g |
| Cropovidone | 5 g |

1000 tablets (weight: 100 mg per tablet) are produced.

Preparation method is as follows:

The crystal entecavir, sodium lauryl sulfate and sucrose fatty acid ester are respectively sieved through 120 mesh screen and uniformly mixed to obtain a mixture, then the mixture is uniformly mixed with mannitol, dextrin, alginic acid, sodium alginate and crospovidone, and then filled into a No. 4 size of capsule.

### Comparative Example 1

| Ingredients: | |
|---|---|
| Amorphous entecavir | 0.001 g |
| Stearic acid | 1.999 g |
| Sucrose | 50 g |
| Calcium sulfate | 40 g |
| Poloxamer | 3 g |
| Hydroxypropyl cellulose | 5 g |

1000 tablets (weight: 100 mg per tablet) are produced.

Preparation method is as follows:

The amorphous entecavir and stearic acid are respectively sieved through 120 mesh screen and uniformly mixed to obtain a mixture, then the mixture is uniformly mixed with sucrose, calcium sulfate, poloxamer and hydroxypropyl cellulose, and then compressed into a tablet.

### Comparative Example 2

| Ingredients: | |
|---|---|
| Amorphous entecavir | 0.01 g |
| Silica | 0.05 g |
| Magnesium stearate | 0.05 g |
| Lactose | 89.89 g |
| Povidone | 5 g |
| Sodium carboxymethyl starch | 5 g |

1000 tablets (weight: 100 mg per tablet) are produced.

Preparation method is as follows:

The amorphous entecavir, silica and magnesium stearate are respectively sieved through 120 mesh screen and uniformly mixed to obtain a mixture, then the mixture is uniformly mixed with lactose, povidone and sodium carboxymethyl starch, and then compressed into a tablet.

### Comparative Example 3

| Ingredients: | |
|---|---|
| Amorphous entecavir | 0.1 g |
| Sucrose fatty acid ester | 5 g |
| Starch | 81.9 g |
| Alginic acid | 8 g |
| Hydroxypropyl cellulose | 2 g |
| Sodium starch glycolate | 3 g |

1000 tablets (weight: 100 mg per tablet) are produced.

Preparation method is as follows:

The amorphous entecavir and sucrose fatty acid ester are respectively sieved through 120 mesh screen and uniformly mixed to obtain a mixture, then the mixture is uniformly mixed with starch, alginic acid, hydroxypropyl cellucose and sodium starch glycolate, and then compressed into a tablet.

### Comparative Example 4

| Ingredients: | |
|---|---|
| Amorphous entecavir | 0.5 g |
| Sodium stearate | 3 g |
| Lactose | 45 g |
| Starch | 38.5 g |
| Hydroxypropylmethyl cellulose | 10 g |
| Crospovidone | 3 g |

1000 tablets (weight: 100 mg per tablet) are produced.

Preparation method is as follows:

The amorphous entecavir and sodium stearate are respectively sieved through 120 mesh screen and uniformly mixed to obtain a mixture, then the mixture is uniformly mixed with lactose, starch, hydroxypropylmethyl cellulose and crospovidone, and then compressed into a tablet.

### Comparative Example 5

| Ingredients: | |
|---|---|
| Amorphous entecavir | 0.5 g |
| Silica | 1.5 g |
| Magnesium stearate | 1.5 g |
| Lactose | 25 g |
| Starch | 30 g |
| Microcrystalline cellulose | 34.5 g |
| Povidone | 6 g |
| Sodium carboxymethyl starch | 3 g |

1000 tablets (weight: 100 mg per tablet) are produced.

Preparation method is as follows:

The amorphous entecavir, magnesium stearate and silica are respectively sieved through 120 mesh screen and uniformly mixed to obtain a mixture, then the mixture is uniformly mixed with lactose, starch, microcrystalline cellulose, povidone and sodium carboxymethyl starch, and then compressed into a tablet.

### Comparative Example 6

| Ingredients: | |
|---|---|
| Amorphous entecavir | 1 g |
| Silica | 1.5 g |
| Magnesium stearate | 1.5 g |
| Starch | 84 g |
| Povidone | 10 g |
| Sodium carboxymethyl starch | 2 g |

1000 tablets (weight: 100 mg per tablet) are produced.

Preparation method is as follows:

The amorphous entecavir, silica and magnesium stearate are respectively sieved through 120 mesh screen and uniformly mixed to obtain a mixture, then the mixture is uniformly mixed with starch, povidone and sodium carboxymethyl starch, and then compressed into a tablet.

### Comparative Example 7

| Ingredients: | |
|---|---|
| Amorphous entecavir | 1.5 g |
| Sodium lauryl sulfate | 5 g |
| Microcrystalline cellulose | 87.5 g |
| Carbomer | 4 g |
| Croscarmellose sodium | 2 g |

1000 tablets (weight: 100 mg per tablet) are produced.

Preparation method is as follows:

The amorphous entecavir and sodium lauryl sulfate are respectively sieved through 120 mesh screen and uniformly mixed to obtain a mixture, then the mixture is uniformly mixed with microcrystalline cellulose, carbomer and croscarmellose sodium, and then compressed into a tablet.

### Comparative Example 8

| Ingredients: | |
|---|---|
| Amorphous entecavir | 2 g |
| Magnesium stearate | 3 g |
| Lactose | 38 g |
| Microcrystalline cellulose | 50 g |
| Povidone | 2 g |
| Sodium carboxymethyl starch | 5 g |

1000 tablets (weight: 100 mg per tablet) are produced.

Preparation method is as follows:

The amorphous entecavir and magnesium stearate are respectively sieved through 120 mesh screen and uniformly mixed to obtain a mixture, then the mixture is uniformly mixed with lactose, microcrystalline cellulose, povidone and sodium carboxymethyl starch, and then compressed into a tablet.

### Comparative Example 9

| Ingredients: | |
|---|---|
| Amorphous entecavir | 5 g |
| Silica | 5 g |
| Microcrystalline cellulose | 71 g |
| Povidone | 18 g |
| Sodium carboxymethyl starch | 1 g |

1000 tablets (weight: 100 mg per tablet) are produced.

Preparation method is as follows:

The amorphous entecavir and silica are respectively sieved through 120 mesh screen and uniformly mixed to obtain a mixture, then the mixture is uniformly mixed with microcrystalline cellulose, povidone and sodium carboxymethyl starch, and then compressed into a tablet.

### Comparative Example 10

| Ingredients: | |
|---|---|
| Amorphous entecavir | 10 g |
| Sodium stearate | 2 g |
| Talcum powder | 2 g |
| Glucose | 30 g |
| Calcium phosphate | 40 g |
| Hydroxypropylmethyl cellulose | 11 g |
| Sodium starch glycolate | 5 g |

1000 tablets (weight: 100 mg per tablet) are produced.

Preparation method is as follows:

The amorphous entecavir, sodium stearate and talcum powder are respectively sieved through 120 mesh screen and uniformly mixed to obtain a mixture, then the mixture is uniformly mixed with glucose, calcium phosphate, hydroxypropylmethyl cellulose and sodium starch glycolate, and then filled into a No. 4 size of capsule.

### Comparative Example 11

| Ingredients: | |
|---|---|
| Amorphous entecavir | 15 g |
| Stearic acid | 4 g |
| Xylitol | 43 g |
| Maltitol | 30 g |
| Gelatin | 6 g |
| Croscarmellose sodium | 2 g |

1000 tablets (weight: 100 mg per tablet) are produced.

Preparation method is as follows:

The amorphous entecavir and stearic acid are respectively sieved through 120 mesh screen and uniformly mixed to obtain a mixture, then the mixture is uniformly mixed with xylitol, maltitol, gelatin and croscarmellose sodium, and then filled into a No. 4 size of capsule.

### Comparative Example 12

| Ingredients: | |
|---|---|
| Amorphous entecavir | 25 g |
| Sodium lauryl sulfate | 2.5 g |
| Sucrose fatty acid ester | 2.5 g |
| Mannitol | 30 g |
| Dextrin | 20 g |
| Alginic acid | 6 g |
| Sodium alginate | 9 g |
| Cropovidone | 5 g |

1000 tablets (weight: 100 mg per tablet) are produced.

Preparation method is as follows:

The amorphous entecavir, sodium lauryl sulfate and sucrose fatty acid ester are respectively sieved through 120 mesh screen and uniformly mixed to obtain a mixture, then the mixture is uniformly mixed with mannitol, dextrin, alginic acid, sodium alginate and crospovidone, and then filled into a No. 4 size of capsule.

### Example 13

18 tablets of Examples 1-9 and Comparative examples 1-9 are film coated with commercially film coating powder by using conventional Film Coating Techniques. Ingredients of the coating solution is as follows:

| | |
|---|---|
| Opadry ® II | 2 mg~5 mg |
| Pure water | appropriate |

Note:
The pure water used in coating process can be removed by drying.

Opadry®II is commercially film coated premixed powders and contains hydroxypropylmethylcellulose, titanium dioxide, polyethylene glycol, and polysorbate 80, as well as various of color starch according to different standards of the series products. The amount of the coating powder used in the formulation is an average amount range of the coating powder per tablet in an account of 100 mg per tablet.

### Example 14

| Ingredients: | |
|---|---|
| Crystal entecavir | 5 g |
| Lactose | 72 g |
| Hydroxypropylmethyl cellulose | 17 g |
| Total | 94 g |

Preparation method is as follows:

The crystal entecavir, lactose and hydroxypropylmethyl cellulose are respectively sieved through 120 mesh screen and uniformly mixed to obtain a premixed composition comprising crystal entecavir, lactose and hydroxypropylmethyl cellulose.

After these samples produced by the Examples and the Comparative Examples above are respectively stored for 10 days under the conditions of 60° C. of temperature, 92.5% of Relative Humidity (RH) and 45001±5001× of illuminance, the amount of each ingredient in each sample is determined by HPLC (area normalized method). Octadecylsilane bonded silica is used as filler, water-acetonitrile-trifluoroacetic acid (990:10:1) is used as a mobile phase A, and water-acetonitrile-trifluoroacetic acid (700:300:1) is used as a mobile phase B. The flow rate is about 1.0 ml/min, the detecting wavelength is about 254 nm, and the temperature of the column is about 30° C. The result is in Table 1 below:

TABLE 1
Stability of the Examples and the Comparative Examples

| | Related substance (%) | | | | | Related substance (%) | | | |
|---|---|---|---|---|---|---|---|---|---|
| | | 60° C. | 92.5% RH | 4500 lx ± 500 lx | | | 60° C. | 92.5% RH | 4500 lx ± 500 lx |
| Example | 0 day | for 10 days | for 10 days | for 10 days | Comparative Example | 0 day | for 10 days | for 10 days | for 10 days |
| Example 1 | 0.51 | 1.78 | 0.66 | 1.32 | Comparative Example 1 | 0.52 | 2.69 | 1.45 | 2.50 |
| Example 2 | 0.49 | 1.80 | 0.61 | 1.35 | Comparative Example 2 | 0.53 | 2.88 | 1.42 | 2.53 |
| Example 3 | 0.51 | 1.68 | 0.58 | 1.44 | Comparative Example 3 | 0.55 | 2.74 | 1.38 | 2.60 |
| Example 4 | 0.54 | 1.82 | 0.64 | 1.36 | Comparative Example 4 | 0.58 | 2.85 | 1.48 | 2.55 |
| Example 5 | 0.50 | 1.85 | 0.67 | 1.34 | Comparative Example 5 | 0.54 | 2.71 | 1.45 | 2.53 |
| Example 6 | 0.47 | 1.90 | 0.60 | 1.41 | Comparative Example 6 | 0.51 | 2.92 | 1.41 | 2.59 |
| Example 7 | 0.57 | 1.65 | 0.68 | 1.30 | Comparative Example 7 | 0.62 | 2.45 | 1.54 | 2.51 |
| Example 8 | 0.40 | 1.56 | 0.54 | 1.33 | Comparative Example 8 | 0.53 | 2.43 | 1.36 | 2.56 |
| Example 9 | 0.43 | 1.77 | 0.57 | 1.34 | Comparative Example 9 | 0.48 | 2.80 | 1.44 | 2.58 |
| Example 10 | 0.58 | 1.68 | 0.71 | 1.29 | Comparative Example 10 | 0.62 | 2.75 | 1.58 | 2.47 |
| Example 11 | 0.60 | 1.92 | 0.73 | 1.26 | Comparative Example 11 | 0.65 | 2.96 | 1.60 | 2.45 |
| Example 12 | 0.52 | 1.84 | 0.63 | 1.28 | Comparative Example 12 | 0.63 | 2.90 | 1.42 | 2.50 |

Note: the related substance is referred to as “impurity”, its data shows stability of the products. The amount of the related substance in the product for 0 day is a datum quantity of the related substance. After processes of high temperature, high humidity and lighting, the amount of the related substance may vary, and the more amounts of the related substance increases, the poorer stability of the product is under the condition, vice versa.

It can be shown from the Table 1 that the amount of the related substance of the sample by using the crystal entecavir of the Examples do not remarkably differ from that of using the amorphous entecavir of the Comparative Examples after storing for 0 day. However, after processes of 60° C. of high temperature, 92.5% of high Relative Humidity (RH) and 45001±5001× of illuminance, the increased amount of the related substance of the sample in the Comparative Examples is obviously larger than that of the Examples. It shows that the stability of the samples by using crystal entecavir of Examples is superior to that of samples by using amorphous entecavir of the Comparative Examples.

Although the present invention has been described in connection with the above embodiments, it should be understood that the present invention is not limited to such preferred embodiments and procedures set forth above. The embodiments and procedures were chosen and described in order to best explain the principles of the invention and its practical application, to thereby enable others skilled in the art to best utilize the invention. It will be apparent to those skilled in the art that various substitution, modifications and changes may be thereto without departing from the scope and spirit of the invention. Therefore, the intention is intended to cover all alternative constructions and equivalents falling within the spirit and scope of the invention as defined only by the appended claims and equivalents thereto.

1. A pharmaceutical composition for treating hepatitis B virus infection, comprising the crystal entecavir as active ingredients and pharmaceutically acceptable excipients.

2. The pharmaceutical composition according to claim 1, wherein said crystal entecavir is present at an amount of from 0.001 mg to 25 mg.

3. The pharmaceutical composition according to claim 2, wherein said crystal entecavir is present at an amount of from 0.01 mg to 10 mg.

4. The pharmaceutical composition according to claim 3, wherein said crystal entecavir is present at an amount of from 0.01 mg to 5 mg.

5. The pharmaceutical composition according to claim 1, wherein said pharmaceutically acceptable excipients include diluents, in an amount of from 50% to 90% by weight of the total composition, which adheres with the crystal entecavir by an adhesive.

6. The pharmaceutical composition according to claim 5, wherein said diluents include one or more compounds selected from lactose, starch, microcrystalline cellulose, sucrose, glucose, mannitol, xylitol, maltitol, dextrin, calcium sulfate and calcium phosphate.

7. The pharmaceutical composition according to claim 5, wherein said diluents include lactose, starch and microcrystalline cellulose.

8. The pharmaceutical composition according to claim 5, wherein said adhesives include one or more compounds selected from povidone, hydroxypropylmethylcellulose, alginic acid, sodium alginate, carbomer, poloxamer, and gelatin.

9. The pharmaceutical composition according to claim 5, wherein said adhesive includes povidone, which is present in an amount of from 2% to 18% by weight of the total composition.

10. The pharmaceutical composition according to claim 1, further comprising glidants and disintegrants.

11. The pharmaceutical composition according to claim 10, wherein glidants include one or more compounds selected from silica, stearic acid, magnesium stearate, sodium stearate, calcium stearate, sodium lauryl sulfate, sucrose fatty acid ester, and talcum powder.

12. The pharmaceutical composition according to claim 10, wherein glidants includes silica and magnesium stearate, which is present in an amount of from 0.1% to 5% by weight of the total composition.

13. The pharmaceutical composition according to claim 10, wherein said disintegrants include one or more compounds selected from sodium carboxymethyl starch, hydroxypropyl cellulose, croscarmellose sodium, crospovidone, and sodium starch glycolate.

14. The pharmaceutical composition according to claim 10, wherein said disintegrant includes sodium carboxymethyl starch, which is present in an amount of from 1% to 5% by weight of the total composition.

15. The pharmaceutical composition according to claim 1, wherein said pharmaceutical composition is in the forms of tablets and capsules.

16. The pharmaceutical composition according to claim 15, wherein said pharmaceutical composition has an outer film coating.

17. A method of preparing for the pharmaceutical composition according to claim 1, which comprises the following steps:
(1) sieving the crystal entecavir as a pharmaceutically active ingredient and glidant through 120 mesh screen and uniformly mixing them to obtain a mixture;
(2) uniformly mixing the mixture of step (1) with the diluent, adhesive and disintegrant, and then compressing the mixture into tablets or filling into capsules.

18. Use of the pharmaceutical composition according to claim 1 in preparing for pharmaceuticals for treating hepatitis B virus infection.
